(19) United States
(12) Patent Application Publication
(10) Pub. No.: US 2014/0052016 A1
(43) Pub. Date: Feb. 20, 2014

(54) SYSTEM AND METHOD FOR DIAGNOSING AUTISM

(71) Applicant: NATIONAL YANG-MING UNIVERSITY, Taipei City (TW)

(72) Inventor: Ya-Wei CHENG, Taipei City (TW)

(73) Assignee: NATIONAL YANG-MING UNIVERSITY, TAIPEI CITY (TW)

(21) Appl. No.: 13/734,427

(22) Filed: Jan. 4, 2013

(30) Foreign Application Priority Data
Aug. 16, 2012 (TW) 101129761

(51) Int. Cl. A61B 5/0484

(52) U.S. Cl. CPC A61B 5/04845
USPC 600/544

(57) ABSTRACT

A system for diagnosing autism comprises a measuring device, a movie display, a speaker, and a computer connected with the measuring device and the speaker. The computer contains a control program therein, the control program being capable of arranging a sequence of human-voiced syllables in a pseudo-random order and allowing the sequence of the human-voiced syllables to be played from the speaker while a silent subtitled movie being played on the movie display. The measuring device can measure brain response of a patient to obtain an averaged MMN value, which can be compared with a cut-off point by the control program, whereby, if the averaged MMN value is less than the cut-off point, the patient is deemed to have suffered from an autism. The present invention can provide objective data for quantitative analysis for autism, and provide an easy way to diagnose autism.

### FIELD OF THE INVENTION

The present invention relates to a system and method for diagnosing autism, which can provide objective data for quantitative analysis for autism and provide an easy way to diagnose autism.

### BACKGROUND OF THE INVENTION

Autism spectrum disorder (ASD), being simply termed as autism, is a developmental disorder that appears in the first 3 years of life, and affects the brain's normal development of social and communication skills. The exact causes of autism remain unknown. There are various causes that may lead to autism, among which genetic and environmental factors are important. There is no medicine up to now that can treat the underlying problem of autism. According to a recent report of the U.S. Centers for Disease Control and Prevention (CDC), the prevalence of ASDs in the United States is estimated that, every 1000 children, there are 11.3 children having an ASD, which is higher than that in the past. Although autism and related disorders are more common than previously thought, it is unclear whether this is due to an increasing rate of the illness or an increased ability to diagnose the illness.

Currently, there is no biological test for autism, the diagnosis is based on behavioral criteria and conducted by a health care provider or a psychologist to evaluate a person whether or not to have an autism. There are no objective and quantitative data to be used as a basis for diagnosing autism. Besides, the diagnosis is possible for a child with at least an age 2 or 3, which is inconvenient. Thus, there is a need to provide a system and method that can diagnose autism quantitatively and conveniently.

### SUMMARY OF THE INVENTION

An object of the present invention is to provide a system and method for diagnosing autism that can provide objective data for quantitative analysis for diagnosing autism.

Another object of the present invention is to provide a system and method for diagnosing autism that allows automation of diagnosing autism.

According to one aspect, the system for diagnosing autism comprises a measuring device, a movie display, a speaker, and a computer connected with the measuring device and the speaker. The computer contains a control program therein, the control program being capable of arranging a sequence of human-voiced syllables in a pseudo-random order and allowing the sequence of the human-voiced syllables to be played from the speaker while a silent subtitled movie being played on the movie display. The sequence of the human-voiced syllables is selected from a first group of syllables stored in the computer, containing a human-voiced standard syllable, a first human-voiced deviant syllable, and a second human-voiced deviant syllable. The sequence of the human-voiced syllables is scheduled such that at least two human-voiced standard syllables are interspersed between two human-voiced deviant syllables. Accordingly, while the sequence of the human-voiced syllables is being played from the speaker and the silent subtitled movie is being played on the movie display for the patient, the measuring device can measure brain response of a patient to obtain a plurality of mismatch response values, which can be transferred to the computer, on which the mismatch response values can be averaged and compared with a cut-off point by the control program, whereby, if the averaged mismatch response value is less than the cut-off point, the patient is deemed to have suffered from an autism.

According to another aspect of the present invention, the control program is capable of arranging a sequence of complex-synthesized syllables in a pseudo-random order, and allows the sequence of the complex-synthesized syllables to be played from the speaker while a silent subtitled movie is played on the movie display, wherein the sequence of the complex-synthesized syllables is selected from a second group of syllables stored in the computer, which contains a complex-synthesized standard syllable, a first complex-synthesized deviant signal, and a second complex-synthesized deviant signal, the complex-synthesized standard syllable being derived from the human-voiced standard syllable, the first complex-synthesized deviant syllable being derived from the first human-voiced deviant syllable, the second complex-synthesized deviant syllable being derived from the second human-voiced deviant syllable, the complex-synthesized syllables having sound energy substantially the same as the corresponding human-voiced syllables; the sequence of the complex-synthesized syllables is scheduled such that at least two complex-synthesized standard syllables are interspersed between two complex-synthesized deviant syllables. Accordingly, while the sequence of the complex-synthesized syllables is being played from the speaker and the silent subtitled movie is being played on the movie display for the patient, the measuring device can measure brain response of the patient to obtain a plurality of mismatch response values, which can be transferred to the computer, on which the mismatch response values can be averaged by the control program to provide additional data for diagnosing autism.

According to a further aspect of the present invention, the control program is capable of arranging a sequence of simple-synthesized syllables in a pseudo-random order, and allows the sequence of the simple-synthesized syllables to be played from the speaker while a silent subtitled movie is played on the movie display, wherein the sequence of the simple-synthesized syllables is selected from a third group of syllables stored in the computer, which contains a simple-synthesized standard syllable, a first simple-synthesized deviant signal, and a second simple-synthesized deviant signal, the simple-synthesized standard syllable being derived from the human-voiced standard syllable, the first simple-synthesized deviant syllable being derived from the first human-voiced deviant syllable, the second simple-synthesized deviant syllable being derived from the second human-voiced deviant syllable, the simple-synthesized syllables having sound energy substantially the same as the corresponding human-voiced syllables; the sequence of the simple-synthesized syllables is scheduled such that at least two simple-synthesized standard syllables are interspersed between two simple-synthesized deviant syllables. Accordingly, while the sequence of the simple-synthesized syllables is being played from the speaker and the silent subtitled movie is being played on the movie display for the patient, the measuring device can measure brain response of the patient to obtain a plurality of mismatch response values, which can be transferred to the computer, on which the mismatch response values can be averaged by the control program to provide additional data for diagnosing autism.

Other objects, advantages, and novel features of the present invention will become more apparent from the following detailed description when taken in conjunction with the accompanying drawings.

### BRIEF DESCRIPTION OF THE DRAWINGS

FIG. 1 shows a schematic diagram of a system for diagnosing autism according to one embodiment of the present invention.

FIG. 2 shows an electrode set of an EEG of the present invention over a scalp of a patient.

FIG. 3 shows a partial outline of operating steps of a control program of the system of the present invention, wherein a sequence of human-voiced syllables is used as stimulus.

FIG. 4 shows a partial outline of operating steps of a control program of the system of the present invention, wherein a sequence of complex-synthesized syllables is used as stimulus.

FIG. 5 shows a partial outline of operating steps of a control program of the system of the present invention, wherein a sequence of simple-synthesized syllables is used as stimulus.

FIG. 6 shows a schematic diagram for deriving a complex-synthesized syllable from a human-voiced syllable.

FIG. 7 shows a schematic diagram for deriving a simple-synthesized syllable from a human-voiced syllable.

### DETAILED DESCRIPTION OF THE PREFERRED EMBODIMENTS

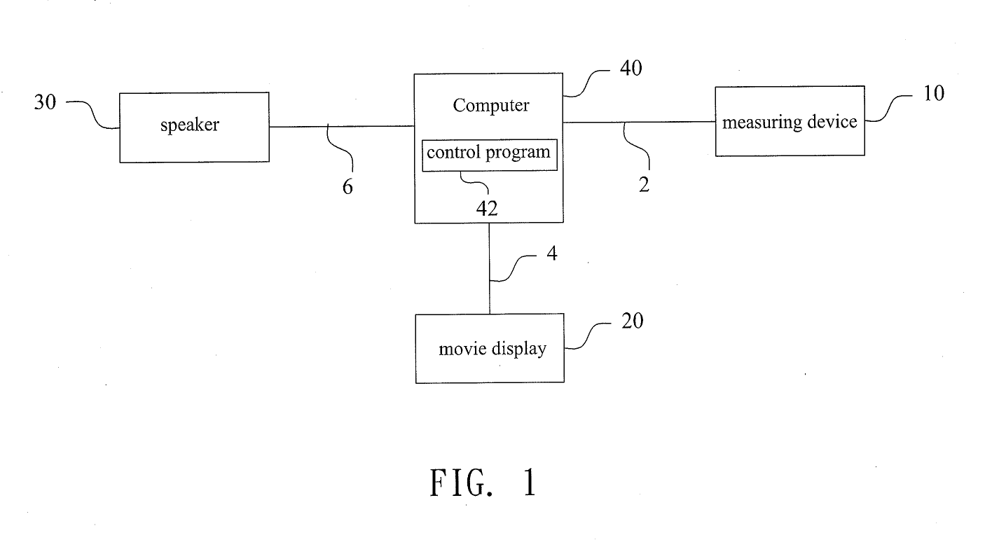

Referring to FIG. 1, a system for diagnosing autism, according to one embodiment of the present invention, is shown. The system generally comprises a measuring device 10, a movie display 20, a speaker 30, and a computer 40. The measuring device 10 is used for measuring brain response of a patient. The computer 40 is connected with the measuring device 10 via a data bus 2, connected with the movie display 20 via a video line 4, and connected with the speaker 30 via an audio line 6. The computer 40 contains a control program 42 therein.

For allowing the system to conduct a diagnosis, the computer 40 needs to be stored with sound data, which includes a first group of syllables, which is recorded from human through a microphone. The first group of syllables contains a human-voiced standard syllable, a first human-voiced deviant syllable, and a second human-voiced deviant syllable, wherein the human-voiced standard syllable is a neutral sound (i.e., a sound without emotion), and the human-voiced deviant syllables are emotional sounds, which may include angry, disgusting, painful, happy, sad, and fearful sound. In this embodiment, the first human-voiced deviant syllable is a happy sound, whereas the second human-voiced deviant syllable is an angry sound.

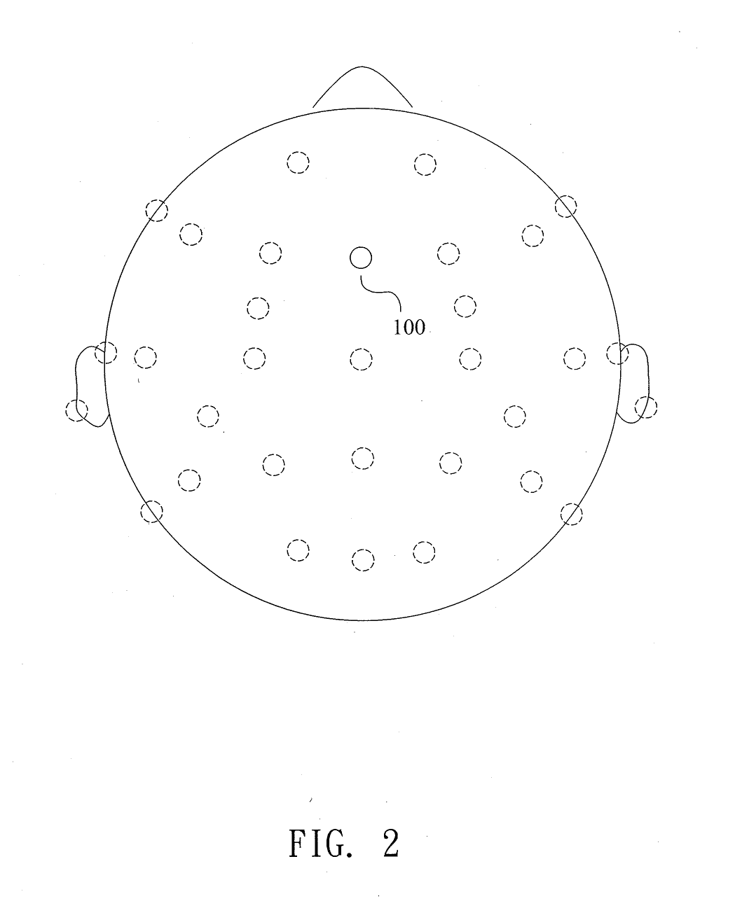

The measuring device 10 can be an EEG (electroencephalograph), which can measure brain wave for a patient. As shown in FIG. 2, the measuring device 10 includes a set of electrodes that can be attached to a patient's scalp, wherein the electrode 100 at the frontal central area of the patient' scalp is a primary concern for obtaining mismatch response (although there are 32 electrodes shown in the figure), which is obtained by subtracting the event-related response to a deviant event from the event-related response to a standard event (i.e., mismatch response =response of standard−response of deviant), being negative in general. In the present invention, the human-voiced standard syllable can serve as a standard event, whereas the first and second human-voiced syllables can serve as a deviant event. For an EEG mismatch response is usually referred as MMN (mismatch negativity), which can reflect the brain's ability to perform automatic comparisons between consecutive stimuli and provide an electrophysiological index of sensory learning. However, MMN has not been used in diagnosing autism yet. In the following paragraphs, a description for autism diagnosis using MMN will be detailed.

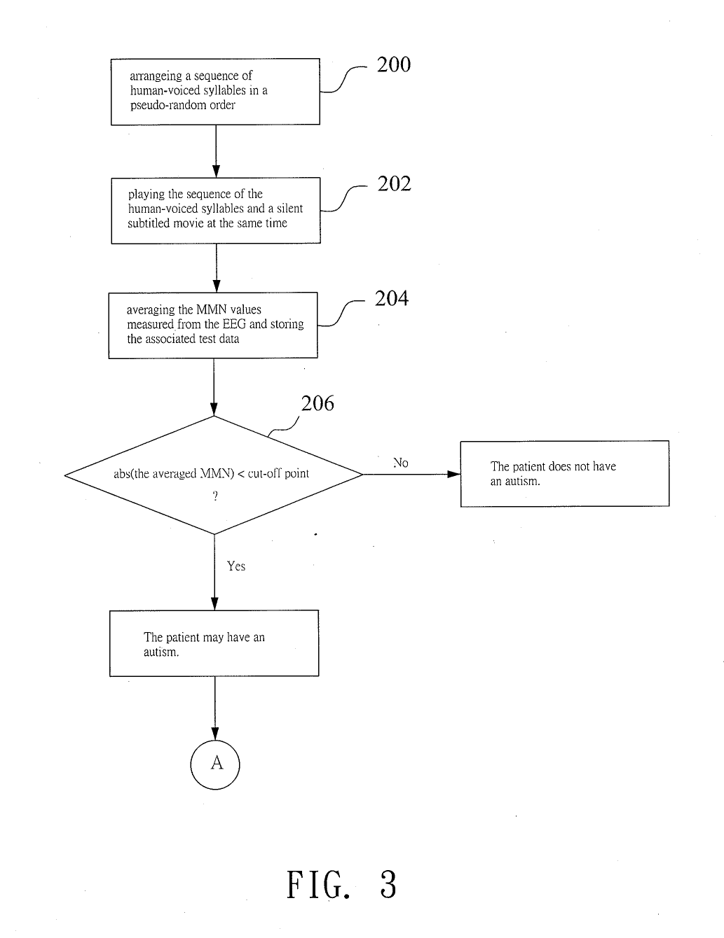

For conducting an autism test, at least, the patient is attached with the electrode 100 of the EEG at the frontal central area of the scalp. The operation of the control program 42 can be divided into several operating steps, as shown in FIG. 3. In step 200, the control program 42 can arrange a sequence of human-voiced syllables in a pseudo-random order. The sequence of the human-voiced syllables is selected from the first group of syllables stored in the computer 40; wherein the total number of the first human-voiced deviant syllables in the sequence of the human-voiced syllables is about 100, the total number of the second human-voiced deviant syllables in the sequence of the human-voiced syllables is about 100, and the total number of the human-voiced standard syllables in the sequence of the human-voiced syllables is about 800; the sequence of the human-voiced syllables is scheduled such that at least two human-voiced standard syllables are interspersed between two human-voiced deviant syllables; the interval between two human-voiced syllables is about 1200 milliseconds. The sequence of the human-voiced syllables serves as stimulus for the autism test.

Next, in step 202, the control program 42 allows the pseudo-random sequence of the human-voiced syllables to be played from the speaker 30 for the patient, and allows a silent subtitled movie to be played on the movie display 20 for the patient at the same time. The purpose of the movie display 20 is to enable the patient not to pay attention to the sound during the test. Thus, the silent subtitled movie can be manually played on an individual video device. Alternatively, an emotionless activity, such as reading or playing game, can be provided for the patient instead of the silent subtitled movie during the test.

Next again, in step 204, the control program 42 can average the measured MMN values from the EEG to obtain an averaged MMN value, which is usually a negative number, for the patient and store the associated test data in the computer 40.

Next again, in step 206, the control program 42 can compare the averaged MMN value with a cut-off point, whereby, if the absolute of the averaged MMN value is less than the cut-off point, the patient may have suffered an autism, or otherwise, the patient does not have an autism. For an adult, the cut-off point can be set to about 2.88 μV, with which the test can reach to a sensitivity of about 95.8% and a specialty of about 91.7% based on ROC analysis. The averaged MMN value can provide an index for quantitative analysis for autism. The less the absolute of the averaged MIVIN value gets, the severer the autism the patient has.

To improve the accuracy of diagnosing autism, the system can be provided with two additional tests, which require the computer 40 being further stored with a second group of syllables and a third group of syllables. The second group of syllables contains a complex-synthesized standard syllable, a first complex-synthesized deviant syllable, and a second complex-synthesized deviant syllable. The third group of syllables contains a simple-synthesized standard syllable, a first simple-synthesized deviant syllable, and a second simple-synthesized deviant syllable.

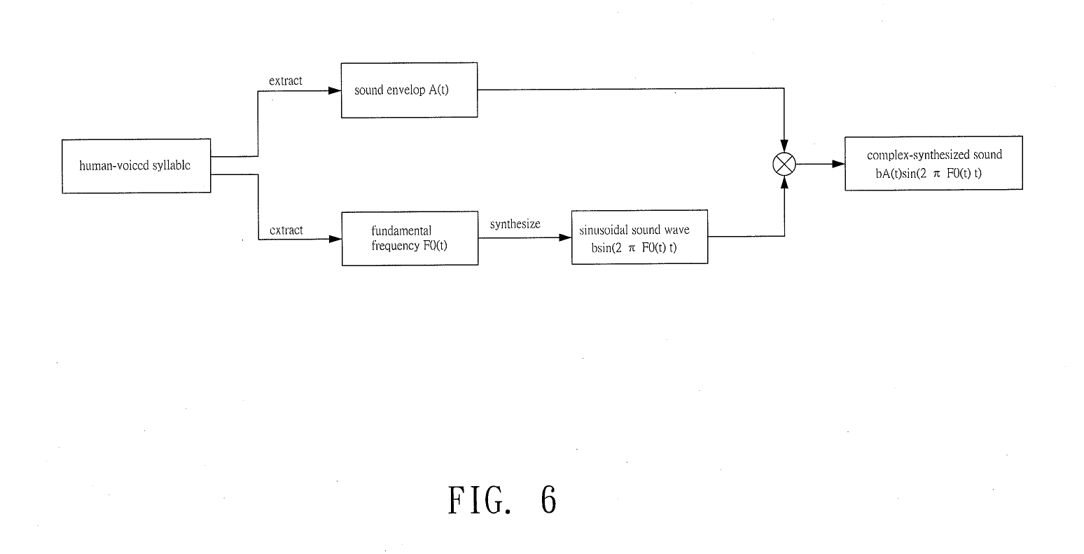

The complex-synthesized syllables can be derived from human-voiced syllables by using acoustic software. FIG. 6 shows a schematic diagram of the synthesis, wherein the sound envelope A(t) and the fundamental frequency F0(t), extracted from a human-voiced syllable, can be combined into a complex-synthesized sound: b*A(t)*sin (2*π*F0(t)*t), wherein t represents time, and b represent a constant. In the synthesis, the sound energy of the complex-synthesized syllable remains substantially the same as that of the human-voiced syllable. In this way, the complex-synthesized standard syllable can be derived from the human-voiced standard syllable, the first complex-synthesized deviant syllable can be derived from the first human-voiced deviant syllable, and the second complex-synthesized deviant syllable can be derived from the second human-voiced deviant syllable. Through the synthesis, some emotional characteristic of the original recorded sound (i.e., the human-voiced syllables) has been removed, however, most physical characteristic of the original recorded sound would remain the same.

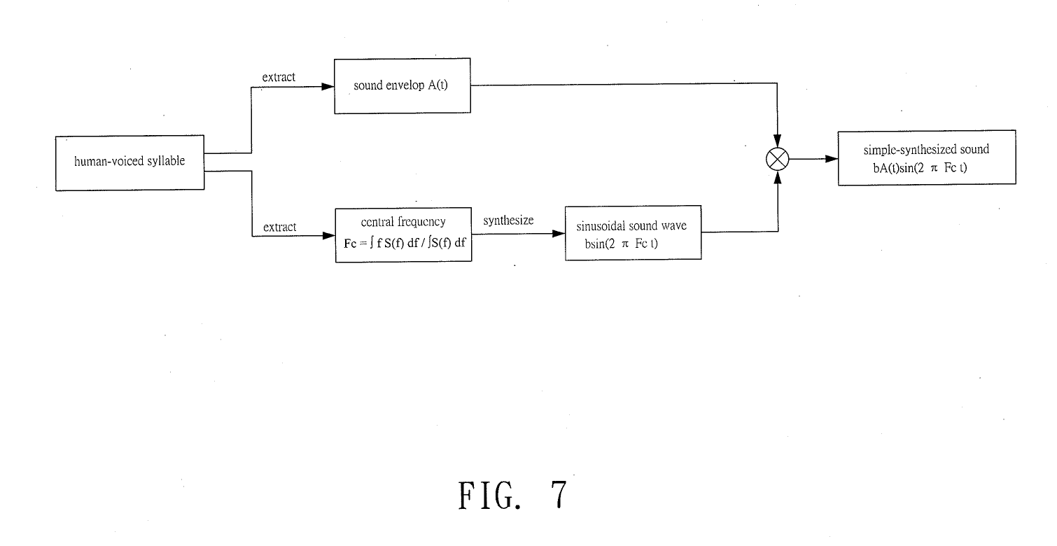

Similarly, the simple-synthesized syllables can be derived from human-voiced syllables by using acoustic software. FIG. 7 shows a schematic diagram of the synthesis, wherein the sound envelope A(t) and the central frequency Fc (=∫*S(f) df/∫S(f) df, S(f) representing the sound spectrum, and f representing frequency), extracted from a human-voiced syllable, can be combined into a simple-synthesized sound: b*sin (2*π*Fc*t), wherein t represents time, and b represents a constant. In the synthesis, the sound energy of the simple-synthesized syllable remains substantially the same as that of the human-voiced syllable. In this way, the simple-synthesized standard syllable can be derived from the human-voiced standard syllable, the first simple-synthesized deviant syllable can be derived from the first human-voiced deviant syllable, and the second simple-synthesized deviant syllable can be derived from the second human-voiced deviant syllable. Through the synthesis, some emotional characteristic of the original recorded sound (i.e., the human-voiced syllables) has been removed, however, most physical characteristic of the original recorded sound would remain the same.

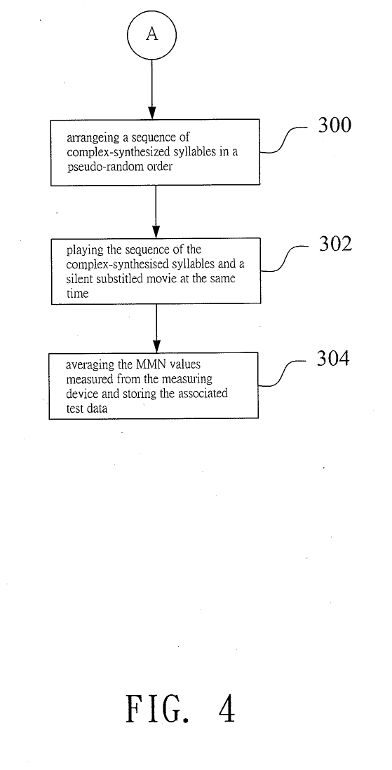

Additional operating steps using complex-synthesized syllables as stimuli can be added for the control program 42, as show in FIG. 4. In step 300, the control program 42 can arrange a sequence of complex-synthesized syllables in a pseudo-random order. The sequence of the complex-synthesized syllables is selected from the second group of syllables stored in the computer 40; wherein the total number of the first complex-synthesized deviant syllables in the sequence of the complex-synthesized syllables is about 100, the total number of the second complex-synthesized deviant syllables in the sequence of the complex-synthesized syllables is about 100, and the total number of the complex-synthesized standard syllables in the sequence of the complex-synthesized syllables is about 800; the sequence of the complex-synthesized syllables is scheduled such that at least two complex-synthesized standard syllables are interspersed between two complex-synthesized deviant syllables; the interval between two complex-synthesized syllables is about 1200 milliseconds.

Next, in step 302, the control program 42 allows the pseudo-random sequence of the complex-synthesized syllables to be played from the speaker 30 for the patient, and allows a silent subtitled movie to be played on the movie display 20 for the patient at the same time. The purpose of the movie display 20 is to enable the patient not to pay attention to the sound during the test. Thus, the silent subtitled movie can be manually played on an individual video device. Alternatively, an emotionless activity, such as reading or playing game, can be provided for the patient instead of the silent subtitled movie during the test.

Next again, in step 304, the control program 42 can average the measured MMN values from the EEG to obtain an averaged MMN value, which is usually a negative number, for the patient and store the associated test data in the computer 40.

A specialist or a health care provider can compare the averaged MMN value with that obtained by using the human-voiced syllables as stimuli. If the absolute of this averaged MMN value is too small, the patient may have suffered a hearing impairment. If the absolute of this averaged MMN value is too large, the patient may have suffered schizophrenia. With this additional test, the specialist or the health care provider can further confirm the patient whether to have suffered an autism or not.

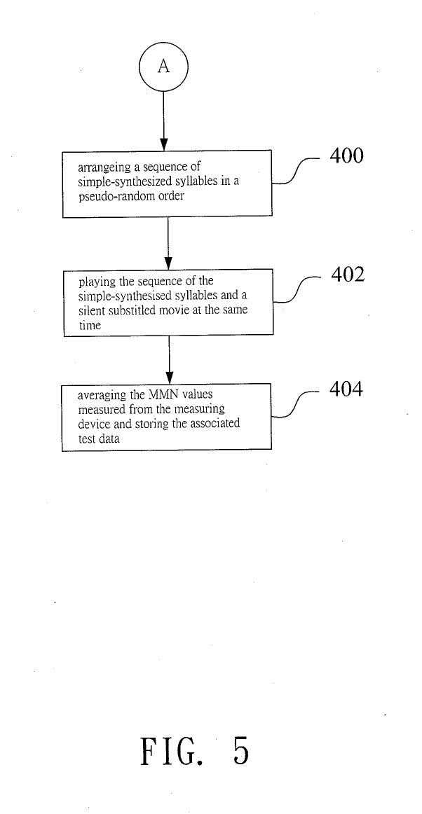

Furthermore, additional operating steps using simple-synthesized syllables as stimuli can be added for the control program 42, as show in FIG. 5. Since the operating steps using simple-synthesized syllables as stimuli are the same as those using complex-synthesized syllables as stimuli, a detailed description for them is eliminated herein. If the absolute of the averaged MMN value is too small, the patient may have suffered a hearing impairment. If the absolute of the averaged MMN value is too large, the patient may have suffered schizophrenia. With the additional test, the specialist or the health care provider can further confirm the patient whether to have suffered an autism or not.

As a summary, the present invention has the following advantages:

1. The MINA values obtained by using human-voiced syllables as stimuli can provide objective data for quantitative analysis for autism, thereby helping a specialist or a health care provider to diagnose a person whether to have an autism or not.

2. With the method of the present invention, the diagnosis for autism can be automated and simplified.

3. The method of the present invention can be conveniently applied to persons with any ages.

Although the present invention has been described with a certain degree of particularity, it is understood that the present disclosure is made by way of example only and the combination and arrangement of parts may be resorted to without departing from the spirit and scope of the invention hereinafter claimed.

I claim:

1. A system for diagnosing autism, comprising:
a device for measuring brain response mainly at a central-frontal area of a patient's scalp;
a movie display;
a speaker; and
a computer being connected with the measuring device via a data bus and the speaker via an audio line, the computer containing a control program therein, the control program being capable of arranging a sequence of human-voiced syllables in a pseudo-random order and allowing the sequence of the human-voiced syllables to be played from the speaker while a silent subtitled movie being played on the movie display, wherein the sequence of the human-voiced syllables is selected from a first group of syllables stored in the computer, which contains a human-voiced standard syllable, a first human-voiced deviant syllable, and a second human-voiced deviant syllable; the sequence of the human-voiced syllables is scheduled such that at least two human-voiced standard syllables are interspersed between two human-voiced deviant syllables; whereby, while the sequence of the human-voiced syllables is being played from the speaker and the silent subtitled movie is being played on the movie display for the patient, the measuring device can measure brain response of the patient to obtain a plurality of mismatch response values, which can be transferred via the data bus to the computer, on which the mismatch response values can be averaged and compared with a cut-off point by the control program, whereby, if the averaged mismatch response value is less than the cut-off point, the patient is deemed to have suffered from an autism.

2. The system of claim 1, wherein the human-voiced standard syllable is a neutral sound, the first human-voiced deviant syllable is a happy sound, and the second human-voiced deviant syllable is an angry sound.

3. The system of claim 2, wherein the total number of the first human-voiced deviant syllables in the sequence of the human-voiced syllables is about 100, the total number of the second human-voiced deviant syllables in the sequence of the human-voiced syllables is about 100, the total number of the human-voiced standard syllables in the sequence of the human-voiced syllables is about 800, and the interval between two human-voiced syllables is about 1200 milliseconds.

4. The system of claim 3, wherein the measuring device is an EEG.

5. The system of claim 1, wherein the control program is capable of arranging a sequence of complex-synthesized syllables in a pseudo-random order, and allows the sequence of the complex-synthesized syllables to be played from the speaker while a silent subtitled movie is played on the movie display, wherein the sequence of the complex-synthesized syllables is selected from a second group of syllables stored in the computer, which contains a complex-synthesized standard syllable, a first complex-synthesized deviant signal, and a second complex-synthesized deviant signal, the complex-synthesized standard syllable being derived from the human-voiced standard syllable, the first complex-synthesized deviant syllable being derived from the first human-voiced deviant syllable, the second complex-synthesized deviant syllable being derived from the second human-voiced deviant syllable, the complex-synthesized syllables having sound energy substantially the same as the corresponding human-voiced syllables; the sequence of the complex-synthesized syllables is scheduled such that at least two complex-synthesized standard syllables are interspersed between two complex-synthesized deviant syllables; whereby, while the sequence of the complex-synthesized syllables is being played from the speaker and the silent subtitled movie is being played on the movie display for the patient, the measuring device can measure brain response of the patient to obtain a plurality of mismatch response values, which can be transferred via the data bus to the computer, on which the mismatch response values can be averaged by the control program to provide additional data for diagnosing autism.

6. The system of claim 1, wherein the control program is capable of arranging a sequence of simple-synthesized syllables in a pseudo-random order, and allows the sequence of the simple-synthesized syllables to be played from the speaker while a silent subtitled movie is played on the movie display, wherein the sequence of the simple-synthesized syllables is selected from a third group of syllables stored in the computer, which contains a simple-synthesized standard syllable, a first simple-synthesized deviant signal, and a second simple-synthesized deviant signal, the simple-synthesized standard syllable being derived from the human-voiced standard syllable, the first simple-synthesized deviant syllable being derived from the first human-voiced deviant syllable, the second simple-synthesized deviant syllable being derived from the second human-voiced deviant syllable, the simple-synthesized syllables having sound energy substantially the same as the corresponding human-voiced syllables; the sequence of the simple-synthesized syllables is scheduled such that at least two simple-synthesized standard syllables are interspersed between two simple-synthesized deviant syllables; whereby, while the sequence of the simple-synthesized syllables is being played from the speaker and the silent subtitled movie is being played on the movie display for the patient, the measuring device can measure brain response of the patient to obtain a plurality of mismatch response values, which can be transferred via the data bus to the computer, on which the mismatch response values can be averaged by the control program to provide additional data for diagnosing autism.

7. In a computer connecting with an EEG and a speaker, a method for causing the computer to conduct a diagnosis operation on a patient wearing an electrode set of the EE(comprising the steps of:
arranging a sequence of human-voiced syllables in a pseudo-random order, wherein the sequence of the human-voiced syllables is selected from a first group of syllables stored in the computer, which contains a human-voiced standard syllable, a first human-voiced deviant signal, and a second human-voiced deviant signal; the sequence of the human-voiced syllables is scheduled such that at least two human-voiced standard syllables are interspersed between two human-voiced deviant syllables;
playing the sequence of the human-voiced syllables from the speaker and providing an emotionless activity for the patient at the same time, while the EEG measuring brain response of the patient to obtain a plurality of MMN values;
averaging the MMN values from the EEG and storing test data in the computer; and
comparing the averaged MMM value with a cut-off point, whereby, if the averaged MMN value is less than the cut-off point, the patient is deemed to have suffered an autism.

8. The method of claim 7, wherein the emotionless activity is a silent subtitled movie played on the movie display.

9. The method of claim 8, wherein the total number of the first human-voiced deviant syllables in the sequence of the human-voiced syllables is about 100, the total number of the second human-voiced deviant syllables in the sequence of the human-voiced syllables is about 100, the total number of the human-voiced standard syllables in the sequence of the human-voiced syllables is about 800, and the interval between two human-voiced syllables is about 1200 milliseconds.

10. The method of claim 9, wherein the human-voiced standard syllable is a neutral sound, the first human-voiced deviant syllable is a happy sound, and the second human-voiced deviant syllable is an angry sound.

11. The method of claim 7, further including the steps of:
arranging a sequence of complex-synthesized syllables in a pseudo-random order, wherein the sequence of the complex-synthesized syllables is selected from a second group of syllables stored in the computer, which contains a complex-synthesized standard syllable, a first complex-synthesized deviant signal, and a second complex-synthesized deviant signal, the complex-synthesized standard syllable being derived from the human-voiced standard syllable, the first complex-synthesized deviant syllable being derived from the first human-voiced deviant syllable, the second complex-synthesized deviant syllable being derived from the second human-voiced deviant syllable, the complex-synthesized syllables having sound energy substantially the same as the corresponding human-voiced syllables; the sequence of the complex-synthesized syllables is scheduled such that at least two complex-synthesized standard syllables are interspersed between two complex-synthesized deviant syllables;
playing the sequence of the complex-synthesized syllables from the speaker and providing an emotionless activity for the patient at the same time, while the EEG measuring brain response of the patient to obtain a plurality of MMN values; and
averaging the MMN values from the EEG and storing test data in the computer;
whereby, the averaged MMN value resulted from the complex-synthesized syllables can further assist a specialist to diagnose autism.

12. The method of claim 11, wherein the total number of the first complex-synthesized deviant syllables in the sequence of the complex-synthesized syllables is about 100, the total number of the second complex-synthesized deviant syllables in the sequence of the complex-synthesized syllables is about 100, the total number of the complex-synthesized standard syllables in the sequence of the complex-synthesized syllables is about 800, and the interval between two complex-synthesized syllables is about 1200 milliseconds.

13. The method of claim 7, further including the steps of:
arranging a sequence of simple-synthesized syllables in a pseudo-random order, wherein the sequence of the simple-synthesized syllables is selected from a third group of syllables stored in the computer, which contains a simple-synthesized standard syllable, a first simple-synthesized deviant signal, and a second simple-synthesized deviant signal, the simple-synthesized standard syllable being derived from the human-voiced standard syllable, the first simple-synthesized deviant syllable being derived from the first human-voiced deviant syllable, the second simple-synthesized deviant syllable being derived from the second human-voiced deviant syllable, the simple-synthesized syllables having sound energy substantially the same as the corresponding human-voiced syllables; the sequence of the simple-synthesized syllables is scheduled such that at least two simple-synthesized standard syllables are interspersed between two simple-synthesized deviant syllables;
playing the sequence of the simple-synthesized syllables from the speaker and providing an emotionless activity for the patient at the same time, while the EEG measuring brain response of the patient to obtain a plurality of MMN values; and
averaging the MMN values from the EEG and storing test data in the computer;
whereby, the averaged MMN value resulted from the simple-synthesized syllables can further assist a specialist to diagnose autism.

14. The method of claim 13, wherein the total number of the first simple-synthesized deviant syllables in the sequence of the simple-synthesized syllables is about 100, the total number of the second simple-synthesized deviant syllables in the sequence of the simple-synthesized syllables is about 100, the total number of the simple-synthesized standard syllables in the sequence of the simple-synthesized syllables is about 800, and the interval between two simple-synthesized syllables is about 1200 milliseconds.
